(19) United States
(12) Patent Application Publication
(10) Pub. No.: US 2005/0148561 A1
(43) Pub. Date: Jul. 7, 2005

(54) Novel triterpene derivatives, preparation thereof and use thereof

(75) Inventors: Carl T. Wild, Gaithersburg, MD (US); Gary N. Robinson, Gaithersburg, MD (US); Mark Ashton, Oxford (GB); Russell Thomas, Siena (IT)

Correspondence Address: STERNE, KESSLER, GOLDSTEIN & FOX PLLC, 1100 NEW YORK AVENUE, N.W., WASHINGTON, DC, 20005, US

(73) Assignee: Panacos Pharmaceuticals, Inc., Gaithersburg, MD (US)

(21) Appl. No.: 10/949,875

(22) Filed: Sep. 27, 2004

Related U.S. Application Data
(60) Provisional application No. 60/559,358, filed on Apr. 5, 2004.
(60) Provisional application No. 60/505,899, filed on Sep. 26, 2003.

(51) Int. Cl. A61K 38/20; A61K 38/21; A61K 31/57; A61K 31/7072; A61K 31/7076; A61K 31/551; A61K 31/513; A61K 31/522; C07J 1/00; C07J 53/00

(52) U.S. Cl. 514/171; 514/559; 514/220; 424/85.2; 514/263.31; 514/43; 424/85.6; 552/511; 552/623; 562/498; 514/252.13; 514/562; 514/269; 514/662; 514/394

(57) ABSTRACT

The present invention relates to novel synthetic derivatives of triterpenes and the use of such derivatives as pharmaceuticals. The present invention is directed to novel compounds of Formula I: 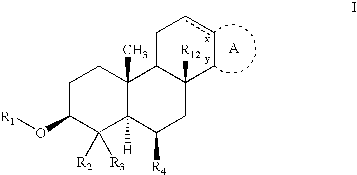
or pharmaceutically acceptable salt or ester thereof, wherein R₁ is a carboxyalkanoyl, where the alkanoyl chain can be interrupted by a nitrogen, sulfur or oxygen atom, or combinations thereof.

This application claims the benefit of U.S. Provisional Application No. 60/505,899, filed Sep. 26, 2003, and U.S. Provisional Application No. 60/559,358, filed Apr. 5, 2004, the entirety of which both are fully incorporated by reference herein.

### BACKGROUND OF THE INVENTION

1. Field of the Invention

The present invention relates to novel synthetic derivatives of triterpenes and the use of such derivatives as pharmaceuticals.

2. Related Art

Retroviruses are small, single-stranded positive-sense RNA viruses. A retroviral particle comprises two identical single-stranded positive sense RNA molecules. Their genome contains, among other things, the sequence of the RNA-dependent DNA polymerase, also known as reverse transcriptase. Many molecules of reverse transcriptase are found in close association with the genomic RNA in the mature viral particles. Upon entering a cell, this reverse transcriptase produces a double-stranded DNA copy of the viral genome, which is then inserted into the chromatin of a host cell. Once inserted, the viral sequence is called a provirus. Retroviral integration is directly dependent upon viral proteins. Linear viral DNA termini (the LTRs) are the immediate precursors to the integrated proviral DNA. There is a characteristic duplication of short stretches of the host's DNA at the site of integration.

Progeny viral genomes and mRNAs are transcribed from the inserted proviral sequence by host cell RNA polymerase in response to transcriptional, regulatory signals in the terminal regions of the proviral sequence, the long terminal repeats, or LTRs. The host cell's protein production machinery is used to produce viral proteins, many of which are inactive until processed by virally encoded proteases. Typically, progeny viral particles bud from the cell surface in a non-lytic manner. Retroviral infection does not necessarily interfere with the normal life cycle of an infected cell or organism. However, neither is it always benign with respect to the host organism. While most classes of DNA viruses can be implicated in tumorigenesis, retroviruses are the only taxonomic group of RNA viruses that are oncogenic. Various retroviruses, such as the Human Immunodeficiency Virus (HIV), which is the etiological agent responsible for acquired immune deficiency syndrome (AIDS) in humans, are also responsible for several very unusual diseases of the immune system of higher animals.

Human Immunodeficiency Virus (HIV) is a member of the lentiviruses, a subfamily of retroviruses. HIV infects and invades cells of the immune system; it breaks down the body's immune system and renders the patient susceptible to opportunistic infections and neoplasms. The immune defect appears to be progressive and irreversible, with a high mortality rate that approaches 100% over several years.

HIV-1 is trophic and cytopathic for T4 lymphocytes, cells of the immune system which express the cell surface differentiation antigen CD4, also known as OKT4, T4 and leu3. The viral tropism is due to the interactions between the viral envelope glycoprotein, gp120, and the cell-surface CD4 molecules (Dalgleish et al., Nature 312: 763-767 (1984)). These interactions not only mediate the infection of susceptible cells by HIV, but are also responsible for the virus-induced fusion of infected and uninfected T cells. This cell fusion results in the formation of giant multinucleated syncytia, cell death, and progressive depletion of CD4 cells in HIV-infected patients. These events result in HIV-induced immunosuppression and its subsequent sequelae, opportunistic infections and neoplasms.

In addition to CD4+ T cells, the host range of HIV includes cells of the mononuclear phagocytic lineage (Dalgleish et al., supra), including blood monocytes, tissue macrophages, Langerhans cells of the skin and dendritic reticulum cells within lymph nodes. HIV is also neurotropic, capable of infecting monocytes and macrophages in the central nervous system causing severe neurologic damage. Macrophage/monocytes are a major reservoir of HIV. They can interact and fuse with CD4-bearing T cells, causing T cell depletion and thus contributing to the pathogenesis of AIDS.

Considerable progress has been made in the development of drugs for HIV-1 therapy during the past few years. Therapeutic agents for HIV can include, but are not limited to, at least one of AZT, 3TC, ddC, d4T, ddI, tenofovir, abacavir, nevirapine, delavirdine, emtricitabine, efavirenz, saquinavir, ritonavir, indinavir, nelfinavir, lopinavir, amprenavir, fosamprenavir, and atazanavir or any other antiretroviral drugs or antibodies in combination with each other, or associated with a biologically based therapeutic, such as, for example, gp41-derived peptides enfuvirtide (Fuzeon; Timeris-Roche) and T-1249 (Trimeris), or soluble CD4, antibodies to CD4, and conjugates of CD4 or anti-CD4, or as additionally presented herein. Combinations of these drugs are particularly effective and can reduce levels of viral RNA to undetectable levels in the plasma and slow the development of viral resistance, with resulting improvements in patient health and life span.

Despite these advances, there are still problems with the currently available drug regimens. Many of the drugs exhibit severe toxicities, have other side-effects (e.g., fat redistribution) or require complicated dosing schedules that reduce compliance and thereby limit efficacy. Resistant strains of HIV often appear over extended periods of time even on combination therapy. The high cost of these drugs is also a limitation to their widespread use, especially outside of developed countries.

There is still a major need for the development of additional drugs to circumvent these issues. Ideally these would target different stages in the viral life cycle, adding to the armamentarium for combination therapy, and exhibit minimal toxicity, yet have lower manufacturing costs.

Previously, betulinic acid and platanic acid were isolated as anti-HIV principles from Syzigium claviflorum. Betulinic acid and platanic acid exhibited inhibitory activity against HIV-1 replication in H9 lymphocyte cells with EC₅₀ values of 1.4 μM and 6.5 μM, respectively, and T.I. values of 9.3 and 14, respectively. Hydrogenation of betulinic acid yielded dihydrobetulinic acid, which showed slightly more potent anti- HIV activity with an EC₅₀ value of 0.9 and a T.I. value of 14 (Fujioka, T., et al., J Nat. Prod. 57: 243-247 (1994)).

Esterification of betulinic acid with certain substituted acyl groups, such as 3′,3′-dimethylglutaryl and 3′,3′-dimethylsuccinyl groups produced derivatives having enhanced activity (Kashiwada, Y., et al., J. Med. Chem. 39: 1016-1017 (1996)). Acylated betulinic acid and dihydrobetulinic acid derivatives that are potent anti-HIV agents are also described in U.S. Pat. No. 5,679,828.

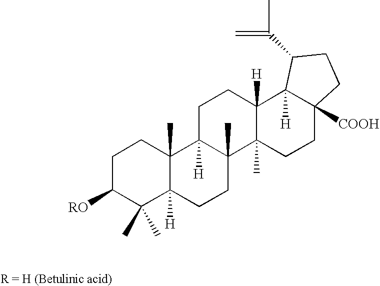

U.S. Pat. No. 5,468,888 discloses 28-amido derivatives of lupanes that are described as having a cytoprotecting effect for HIV-infected cells.

Japanese Patent Application No. JP 01 143,832 discloses that betulin and 3,28-diesters thereof are useful in the anti-cancer field.

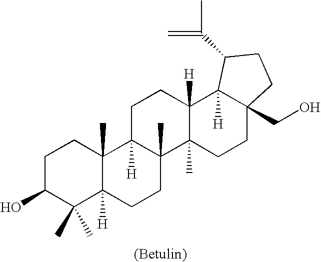

U.S. Pat. No. 6,172,110 discloses betulin and dihydrobetulin derivatives found to have potent anti-HIV activity.

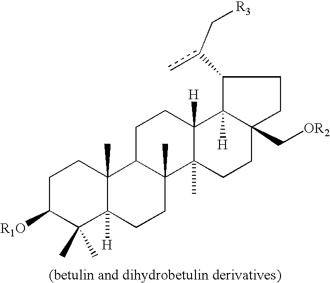

Esterification of the 3 carbon of betulin with succinic acid produced a compound capable of inhibiting HIV-1 activity (Pokrovskii, A. G. et al., Gos. Nauchnyi Tsentr Virusol. Biotekhnol. “Vector” 9: 485-491 (2001)).

Published International Appl. No. WO 02/26761 discloses the use of betulin and analogs thereof for treating fungal infections. U.S. Pat. No. 6,369,101 discloses the use of allobetulin derivatives to treat herpes virus infection. U.S. Pat. No. 3,903,089 discloses the use of ursolic acid derivatives as anti-inflammatory compounds.

A need continues to exist for compounds which possess potent antiretroviral activity, especially anti-HIV activity, with improved biodistribution properties and different modes of action. Such compounds are urgently needed to add to existing anti-HIV therapies. There is also a need for safe and effective compounds that can be topically applied to vaginal or other mucosa to prevent HIV infections between individuals.

### SUMMARY OF THE INVENTION

A first aspect of the present invention is directed to novel compounds of Formula I 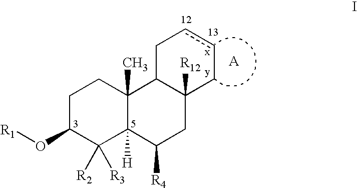
or pharmaceutically acceptable salt or ester thereof;
wherein A is a fused ring of formula 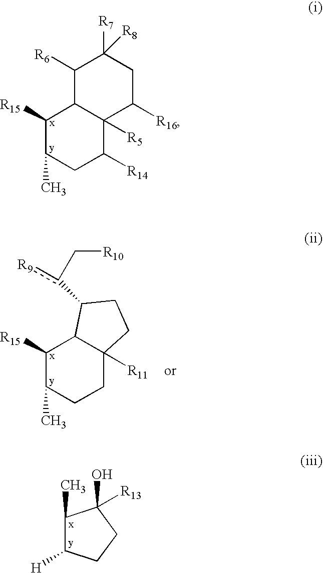
wherein the ring carbons designated x and y in the formulas of A are the same as the ring carbons designated x and y in Formula I;
R₁ is carboxyalkanoyl, where the alkanoyl chain can be optionally substituted by one or more hydroxyl or halo, or can be interrupted by a nitrogen, sulfur or oxygen atom, or combinations thereof;
R₂ and R₃ are independently hydrogen, methyl, halogen, hydroxyl, carboxyl, or —COOR₁₇;
R₄ is hydrogen, methyl, halogen, or hydroxyl;
R₅ is carboxyalkoxycarbonyl, alkoxycarbonyl, alkanoyloxymethyl, carboxyalkanoyloxymethyl, alkoxymethyl, carboxyalkoxymethyl, aminocarbonyl, alkylaminocarbonyl, dialkylaminocarbonyl, alkoxyalkylaminocarbonyl, alkoxyalkoxyalkylaminocarbonyl, alkoxycarbonylaminoalkoxyalkylaminocarbonyl, alkoxycarbonylaminoalkylaminocarbonyl, alkylcarbonylaminoalkylaminocarbonyl, aminoalkylaminocarbonyl, aminoalkoxyalkylaminocarbonyl, monoalkylaminoalkylaminocarbonyl, dialkylaminoalkylaminocarbonyl, heterocyclylcarbonyl, heterocyclylalkylaminocarbonyl, cycloalkylaminocarbonyl, arylaminocarbonyl, arylalkylaminocarbonyl, arylcarbonylaminoalkylaminocarbonyl, or heteroarylaminocarbonyl, any of which is optionally substituted by one or more hydroxyl or halo, or R₅ is a carboxyl or hydroxymethyl, or when either R₂ or R₃ are carboxyl, then R₅ can be methyl;
R₆ is hydrogen, methyl, hydroxyl or halogen;
R₇ and R₈ are independently hydrogen or C₁₋₆ alkyl;
R₉ is CH₂ or CH₃;
R₁₀ is hydrogen, hydroxyl or methyl;
R₁₁, is methyl, methoxycarbonyl, carboxyalkoxycarbonyl, alkanoyloxymethyl, alkoxymethyl or carboxyalkoxymethyl, any of which is optionally substituted by one or more hydroxyl or halo;
R₁₂ is hydrogen or methyl;
R₁₃ is hydrogen or methyl;
R₁₄ is hydrogen or hydroxyl;
R₁₅ is hydrogen if C12 and C13 form a single bond, or R₁₅ is absent if C12 and C13 form a double bond;
R₁₆ is hydrogen or hydroxyl;
R₁₇ is alkyl or carboxyalkyl, wherein the alkyl chain can be optionally substituted by one or more hydroxyl or halo, or can be interrupted by a nitrogen, sulfur or oxygen atom, or combinations thereof; and
wherein the straight dashed line represents an optional double bond between C12 and C13 or C20 and C29;
with the proviso that when A is 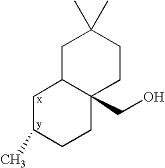
   then R₁ cannot be glutaryl or succinyl when a double bond exists between C12 and C13;
when A is (ii) and R₁₁ is methyl, then R₁ cannot be succinyl;
when A is (iii) and R₂, R₃ and R₁₃ are each hydrogen, then R₁ cannot be succinyl; and
with the proviso that A (i) cannot be 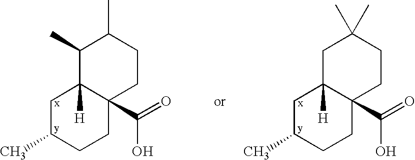
   when R₂ and R₃ are both methyl and a double bond exists between C12 and C13.

In some embodiments, R₁ is selected from the group consisting of:

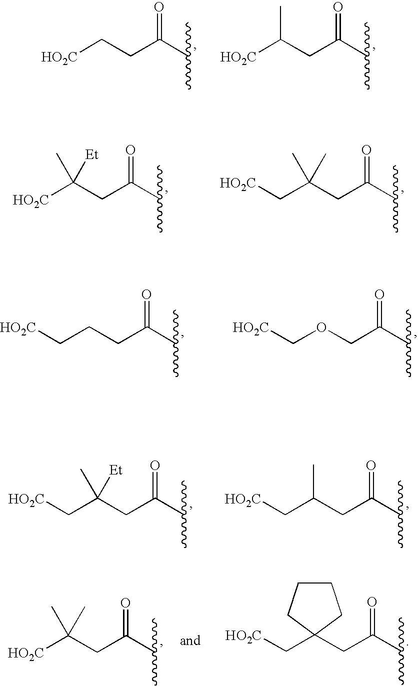

A second aspect of the present invention is directed to pharmaceutical compositions, comprising one or more compounds of Formula I, and a pharmaceutically acceptable carrier or diluent. One or more additional pharmaceutically active compounds can also be included in these compositions.

The compounds of Formula I are useful as anti-retroviral agents. Therefore, the present invention provides methods for inhibiting a retroviral infection in cells or tissue of an animal, comprising administering an effective retroviral inhibiting amount of a compound of Formula I Some embodiments are directed to a method for treating a patient suffering from a retroviral-related pathology, comprising administering to said subject a retroviral inhibiting effective amount of a pharmaceutical composition that includes a compound of Formula I.

The triterpene derivatives of Formula I can be used in a combination therapy with one or more anti-viral agents. Thus, the present invention provides a method of treating a patient suffering from a retroviral-related pathology, comprising administering to said patient a retroviral inhibiting effective amount of at least one compound of Formula I in combination with one or more anti-viral agents. In some embodiments, the anti-viral agent is approved for use for HIV-therapy in the U.S. The present invention is also directed to a method for treating a subject infected with HIV-1 by administering at least one of the above-noted triterpene derivatives, optionally in combination with any one or more of the known anti-AIDS therapeutics or an immunostimulant.

The present invention also provides a method of preventing transmission of HIV infection between individuals. In particular, the present invention provides a method of preventing transmission of HIV infection from an HIV infected pregnant woman to a fetus, comprising administering to said woman and/or said fetus a retroviral inhibiting effective amount of one or more compounds of Formula I during pregnancy or immediately prior to, at, or subsequent to birth.

Further, the present invention provides a method of preventing transmission of HIV infection during sexual intercourse, comprising applying a retroviral inhibiting effective amount of a topical composition including one or more compounds of Formula I to vaginal or other mucosa prior to sexual intercourse.

Furthermore, the present invention is directed to a method for making compounds of Formula I.

Additional embodiments and advantages of the invention will be set forth in part in the description as follows, and in part will be obvious from the description, or can be learned by practice of the invention. The embodiments and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

### DETAILED DESCRIPTION

The compounds of the present invention have the general Formula I: 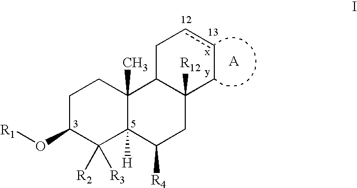
a pharmaceutically acceptable salt or ester thereof:
wherein A is a fused ring of formula 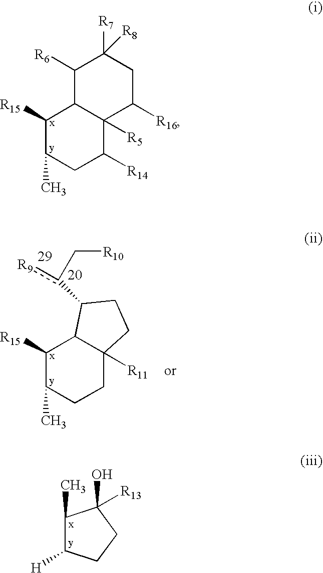
wherein the ring carbons designated x and y in the formulas of A are the same as the ring carbons designated x and y in Formula I;
R₁ is carboxyalkanoyl, where the alkanoyl chain can be optionally substituted by one or more hydroxyl or halo, or can be interrupted by a nitrogen, sulfur or oxygen atom, or combinations thereof;
R₂ and R₃ are independently hydrogen, methyl, halogen, hydroxyl, carboxyl, or COOR₁₇;
R₄ is hydrogen, methyl, halogen, or hydroxyl;
R₅ is carboxyalkoxycarbonyl, alkoxycarbonyl, alkanoyloxymethyl, carboxyalkanoyloxymethyl, alkoxymethyl or carboxyalkoxymethyl, any of which is optionally substituted by one or more hydroxyl or halo, or R₅ is a carboxyl or hydroxymethyl, or when either R₂ or R₃ are carboxyl, then R₅ can be methyl;
R₆ is hydrogen, methyl, hydroxyl or halogen;
R₇ and R₈ are independently hydrogen or C₁₋₆ alkyl;
R₉ is CH₂ or CH₃;
R₁₀ is hydrogen, hydroxyl or methyl;
R₁₁ is methyl, methoxycarbonyl, carboxyalkoxycarbonyl, alkanoyloxymethyl, alkoxymethyl or carboxyalkoxymethyl, any of which is optionally substituted by one or more hydroxyl or halo;
R₁₂ is hydrogen or methyl;
R₁₃ is hydrogen or methyl;
R₁₄ is hydrogen or hydroxyl;
R₁₅ is hydrogen if C12 and C13 form a single bond, or R₁₅ is absent if C12 and C13 form a double bond;
R₁₆ is hydrogen or hydroxyl;
R₁₇ is alkyl or carboxyalkyl, where the alkyl chain can be optionally substituted by one or more hydroxyl or halo, or can be interrupted by a nitrogen, sulfur or oxygen atom, or combinations thereof; and
wherein the straight dashed line represents an optional double bond between C12 and C13 or C20 and C29;
with the proviso that when A is 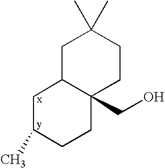
   then R₁ cannot be glutaryl or succinyl when a double bond exists between C12 and C13;
when A is (ii) and R₁₁ is methyl, then R₁ cannot be succinyl;
when A is (iii) and R₂, R₃ and R₁₃ are each hydrogen, then R₁ cannot be succinyl; and
with the proviso that A (i) cannot be 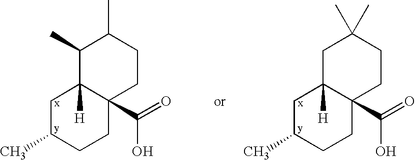
   when R₂ and R₃ are both methyl and a double bond exists between C12 and C13.

In some embodiments, R₁ is a carboxy(C₂₋₁₀)alkylcarbonyl group or a carboxy(C₂₋₁₀)alkoxy(C₁₋₁₀)alkylcarbonyl group. In some embodiments, R₁ is a carboxy(C₂₋₆)alkylcarbonyl group or a carboxy(C₂₋₆)alkoxy(C₁₋₆)alkylcarbonyl group. Suitable R₁ groups are selected from the group consisting of:

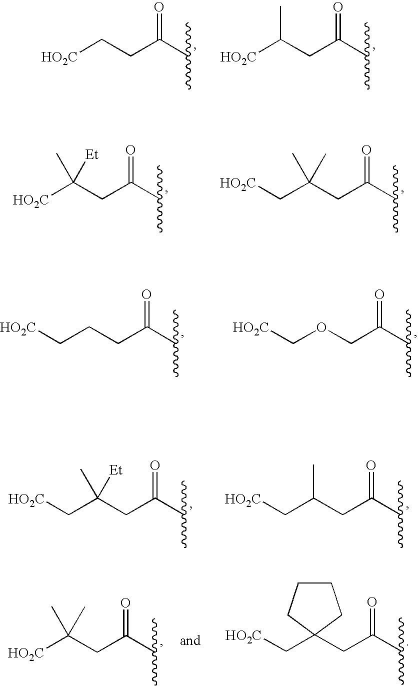

In some embodiments, R₂ and R₃ are independently hydrogen, methyl halogen or hydroxyl. In some embodiments, R₂ and R₃ are independently carboxyl. In some embodiments, R₂ and R₃ are independently COOR₁₇.

In some embodiments, R₁₇ is a carboxy(C₂₋₁₀)alkyl group or a carboxy(C₂₋₁₀)alkoxy(C₁₋₁₀)alkyl group. In some embodiments, R₁₇ is a carboxy(C₂₋₆)alkyl group or a carboxy(C₂₋₆)alkoxy(C₁₋₆)alkyl group. In some embodiments, R₁₇ is selected from the group consisting of:

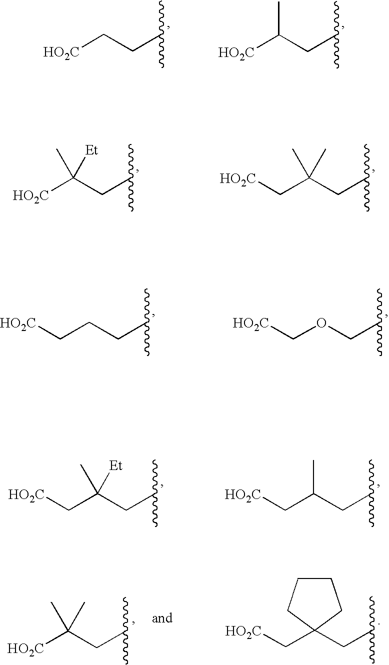

According to the invention, in some embodiments the compounds have Formula II: 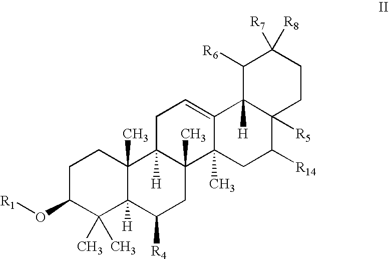
wherein R₁, R₄, R₅, R₆, R₇, R₈ and R₁₄ are as defined above for Formula I. In one embodiment, R₆ is β-methyl, R₈ is hydrogen, R₅ is hydroxymethyl and R₁ is 3′,3′-dimethylglutaryl, 3′,3′-dimethylsuccinyl, glutaryl or succinyl. In another embodiment, R₆ is hydrogen, R₇ and R₈ are both methyl, R₅ is carboxyl and R₁ is 3′,3′-dimethylglutaryl, 3′,3′-dimethylsuccinyl, glutaryl or succinyl.

In some embodiments, R₅ is carboxyalkoxycarbonyl, alkoxycarbonyl, alkanoyloxymethyl, carboxyalkanoyloxymethyl, alkoxymethyl, carboxyalkoxymethyl, aminocarbonyl, alkylaminocarbonyl, dialkylaminocarbonyl, alkoxyalkylaminocarbonyl, alkoxyalkoxyalkylaminocarbonyl, alkoxycarbonylaminoalkoxyalkylaminocarbonyl, alkoxycarbonylaminoalkylaminocarbonyl, alkylcarbonylaminoalkylaminocarbonyl, aminoalkylaminocarbonyl, aminoalkoxyalkylaminocarbonyl, monoalkylaminoalkylaminocarbonyl, dialkylaminoalkylaminocarbonyl, heterocyclylcarbonyl, heterocyclylalkylaminocarbonyl, cycloalkylaminocarbonyl, arylaminocarbonyl, arylalkylaminocarbonyl, arylcarbonylaminoalkylaminocarbonyl, or heteroarylaminocarbonyl, any of which is optionally substituted by one or more hydroxyl or halo, or R₅ is a carboxyl or hydroxymethyl. In some embodiments, R₅ is carboxyalkoxycarbonyl, alkoxycarbonyl, alkanoyloxymethyl, carboxyalkanoyloxymethyl, alkoxymethyl, carboxyalkoxymethyl. In some embodiments, R₅ is selected from a group consisting of carboxyl, hydroxymethyl, —CO₂(CH₂)ₙCOOH, —CO₂(CH₂)ₙCH₃, —CH₂OC(O)(CH₂)ₙCH₃, —CH₂OC(O)(CH₂)ₙCOOH, —CH₂O(CH₂)ₙCH₃ and —CH₂O(CH₂)ₙCOOH. In some embodiments, R₅ is selected from a group consisting of:

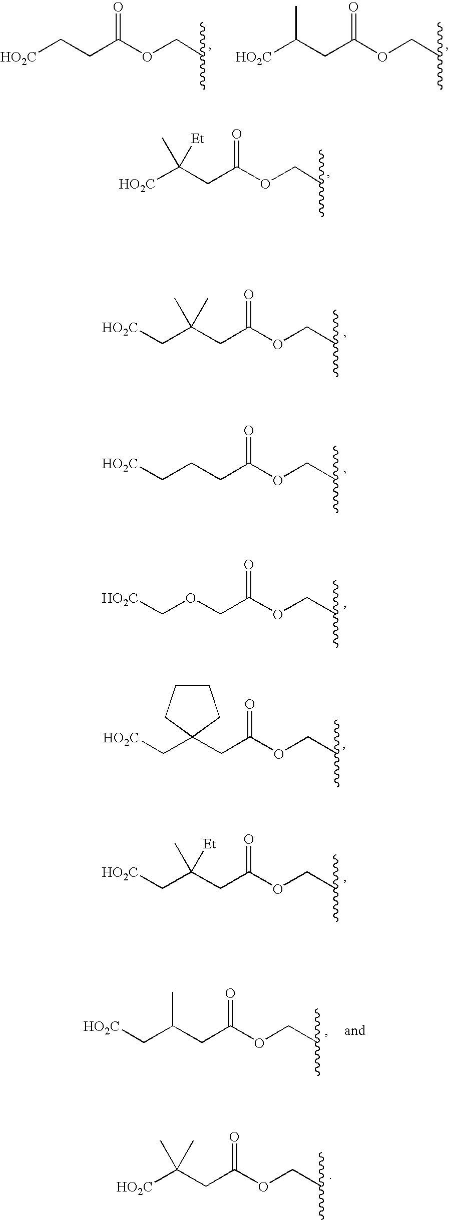

In some embodiments, R₅ is selected from a group consisting of:

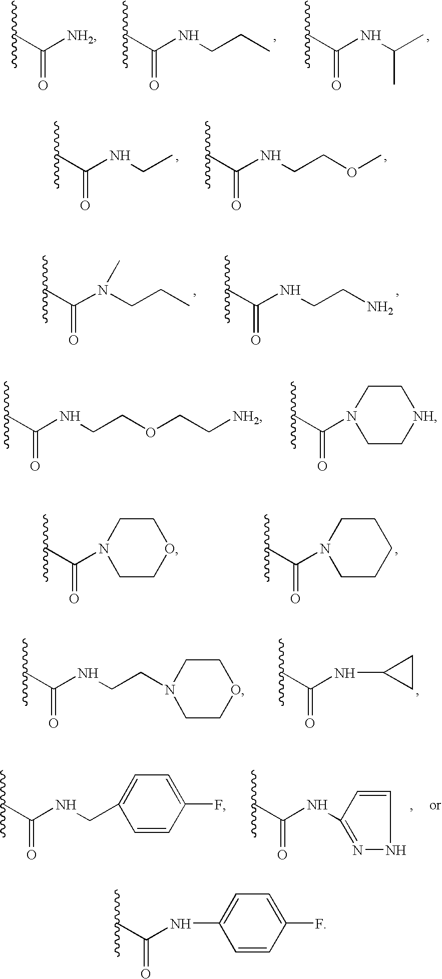

In some embodiments, R₅ is hydroxymethyl. In some embodiments, R₅ is carboxyl. In some embodiments, n is from 0 to 20. In some embodiments, n is from 1 to 10. In some embodiments, n is from 2 to 8. In some embodiments, n is from 1 to 6. In some embodiments, n is from 2 to 6.

In some embodiments, the compounds of the present invention have Formula III: 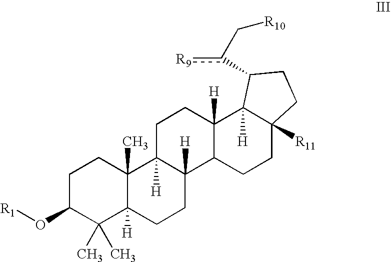
wherein R₁, R₉, R₁₀, and R₁₁ are as defined above for Formula I In one embodiment, R₁ is 3′,3′-dimethylglutaryl, 3′,3′-dimethylsuccinyl, glutaryl or succinyl.

In some embodiments, R₁₁ is methyl, methoxycarbonyl, carboxyalkoxycarbonyl, alkanoyloxymethyl, alkoxymethyl or carboxyalkoxymethyl, any of which is optionally substituted by one or more hydroxyl or halo. In some embodiments, R₁₁ is selected from the group consisting of methyl, —CO₂(CH₂)ₙCOOH, —CH₂OC(O)(CH₂)ₙCH₃, —CH₂O(CH₂)ₙCH₃ and —CH₂O(CH₂)ₙCOOH. In some embodiments, n is from 0 to 20. In some embodiments, n is from 1 to 10. In some embodiments, n is from 2 to 8. In some embodiments, n is from 1 to 6. In some embodiments, n is from 2 to 6. In some embodiments, R₁₁ is methyl. In some embodiments, R₁₁ is methoxycarbonyl. In some embodiments, R₁₁ is selected from the group consisting of methoxymethyl and ethoxymethyl. In some embodiments, methyl groups found in R₁₁ can be substituted with a halogen or a hydroxy.

In some embodiments, the compounds of the present invention have Formula IV: 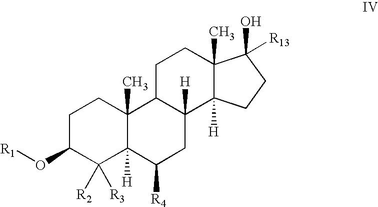
wherein R₁, R₂, R₃, R₄, and R₁₃ are as defined above for Formula I. In one embodiment, R₁ is 3′,3′-dimethylglutaryl, 3′,3′-dimethylsuccinyl, glutaryl or succinyl. In one embodiment, both R₂ and R₃ are methyl.

In some embodiments, the compounds of the present invention have Formula V: 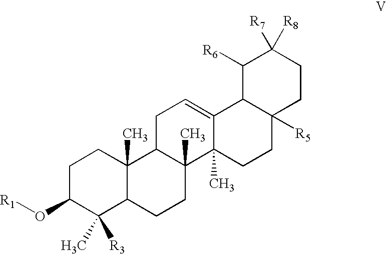
wherein R₁, R₃, R₅, R₆, R₇, and R₈ are as defined as above for Formula I. In some embodiments, R₆ is hydrogen, R₇ is methyl, and R₈ is methyl. In some embodiments, R₆ is methyl, R₇ is hydrogen and R₈ is methyl. In some embodiments, R₃ is carboxyl. In some embodiments, R₃ is COOR₁₇, wherein R₁₇ is defined as above for Formula I.

In some embodiments, the compounds of the present invention have Formula VI: 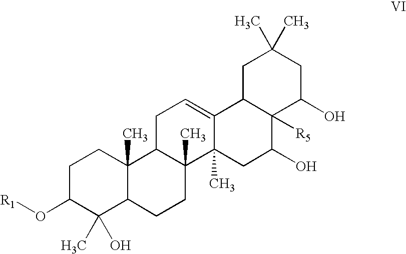
wherein R₁ and R₅ are as defined above for Formula I.

Any triterpene which falls within the scope of Formula I can be used. According to the invention, in some embodiments the compounds of Formula I are selected from the group consisting of derivatives of uvaol, ursolic acid, erythrodiol, echinocystic acid, oleanolic acid, sumaresinolic acid, lupeol, dihydrolupeol, betulinic acid methylester, dihydrobetulinic acid methylester, 17-α-methyl-androstanediol, androstanediol, gymnemic acid, α-boswellic acid, β-boswellic acid and 4,4-dimethyl-androstanediol.

In some embodiments, the compounds of the present invention are defined as in Formula I, wherein R₂ and R₃ are both methyl. In some embodiments, the compounds of the present invention are defined as in Formula I, wherein R₁ is 3′,3′-dimethylsuccinyl. In some embodiments, the compounds of the present invention are defined as in Formula I, wherein R₁ is succinyl, i.e.,

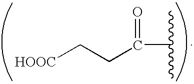

According to the invention, in some embodiments the stereochemistry of the sidechain substituents is important. In some embodiments, the compounds of the present invention are defined as in Formula I, wherein A is (i) and R₅ is in the β position. In some embodiments, the compounds of the present invention are defined as in Formula I, wherein A is (i) and R₆ is in the β position. In some embodiments, the compounds of the present invention are defined as in Formula I, wherein A is (i) and R₁₄ is in the α position. In some embodiments, the compounds of the present invention are defined as in Formula I, wherein A is (i), R₇ is β-methyl, and R₈ is hydrogen. In some embodiments, the compounds of the present invention are defined as in Formula I, wherein A is (i), R₈ is α-methyl, and R₇ is hydrogen. In some embodiments, the compounds of the present invention are defined as in Formula I, wherein A is (i) and both R₇ and R₈ are methyl. In some embodiments, the compounds of the present invention are defined as in Formula I, wherein A is (ii) and R₁₁ is in the β position.

In some embodiments, 3′,3′-dimethylsuccinyl is at the C3 position. In some embodiments, the compounds of Formula II are 3-O-(3′,3′-dimethylsuccinyl)uvaol; 3-O-(3′,3′-dimethylsuccinyl)erythrodiol; 3-O-(3′,3′-dimethylsuccinyl)echinocystic acid or 3-O-(3′,3′-dimethylsuccinyl)sumaresinolic acid. In some embodiments, the compounds of Formula III are 3-O-(3′,3′-dimethylsuccinyl)lupeol; 3-O-(3′,3′-dimethylsuccinyl)dihydrolupeol; 3-O-(3′,3′-dimethylsuccinyl)-17β-methylester-betulinic acid; or 3-O-(3′,3′-dimethylsuccinyl)-17β-methylester-dihydrobetulinic acid. In some embodiments, the compounds of Formula IV are 3-O-(3′,3′-dimethylsuccinyl)-4,4-dimethylandrostanediol; 3-O-(3′,3′-dimethylsuccinyl)-17α-methylandrostanediol; or 3-O-(3′,3′-dimethylsuccinyl)androstanediol. In some embodiments, the compounds of Formula V are 3-O-(3′,3′-dimethylsuccinyl)-α-boswellic acid; or 3-O-(3′,3′-dimethylsuccinyl)-β-boswellic acid. In some embodiments, the compound of Formula VI is 3-O-(3′,3′-dimethylsuccinyl)gymnemic acid.

Alkyl groups and alkyl containing groups of the compounds of the present invention can be straight chain or branched alkyl groups, preferably having one to ten carbon atoms. In some embodiments, the alkyl groups or alkyl containing groups of the present invention can be substituted with a C₃₋₇ cycloalkyl group. In some embodiments, the cycloalkyl group may include, but is not limited to, a cyclobutyl, cyclopentyl or cyclohexyl group.

Also, included within the scope of the present invention are the non-toxic pharmaceutically acceptable salts of the compounds of the present invention. These salts can be prepared in situ during the final isolation and purification of the compounds or by separately reacting the purified compound in its free acid form with a suitable organic or inorganic base and isolating the salt thus formed. These can include cations based on the alkali and alkaline earth metals, such as sodium, lithium, potassium, calcium, magnesium, and the like, as well as nontoxic ammonium, quaternary ammonium and amine cations including, but not limited to ammonium, tetramethylammonium, tetraethylammonium, methylamine, dimethylamine, trimethylamine, ethylamine, N-methyl glucamine and the like.

Also, included within the scope of the present invention are the non-toxic pharmaceutically acceptable esters of the compounds of the present invention. Ester groups are preferably of the type which are relatively readily hydrolyzed under physiological conditions. Examples of pharmaceutically acceptable esters of the compounds of the invention include C₁₋₆ alkyl esters wherein the alkyl group is a straight or branched chain. Acceptable esters also include C₅₋₇ cycloalkyl esters as well as arylalkyl esters, such as, but not limited to benzyl. C₁₋₄ alkyl esters are preferred. In some embodiments, the esters are selected from the group consisting of alkylcarboxylic acid esters, such as acetic acid esters, and mono- or dialkylphosphate esters, such as methylphosphate ester or dimethylphosphate ester. Esters of the compounds of the present invention can be prepared according to conventional methods.

Certain compounds within the scope of Formulae I, II, III, IV, V and VI are derivatives referred to as “prodrugs”. The expression “prodrug” refers to compounds that are rapidly transformed in vivo by an enzymatic or chemical process, to yield the parent compound of the above formulas, for example, by hydrolysis in blood. A thorough discussion is provided by Higuchi, T. and V. Stella in Pro-drugs as Novel Delivery Systems, Vol. 14, A.C.S. Symposium Series, and in Bioreversible Carriers in Drug Design, Ed. Edward B. Roche, American Pharmaceutical Association, Pergamon Press, 1987. Useful prodrugs can be esters of the compounds of Formulae I, II, III, IV, V and VI. In some prodrug embodiments, a lower alkyl group is substituted with one or more hydroxyl or halo groups by a suitable acid. Suitable acids include, e.g., carboxylic acids, sulfonic acids, phosphoric acid or lower alkyl esters thereof, and phosphonic acid or lower alkyl esters thereof. For example, suitable carboxylic acids include alkylcarboxylic acids, such as acetic acid, arylcarboxylic acids and arylalkylcarboxylic acids. Suitable sulfonic acids include alkylsulfonic acids, arylsulfonic acids and arylalkylsulfonic acids. Suitable phosphoric and phosphonic acid esters are methyl or ethyl esters.

In some embodiments, the C3 acyl groups having dimethyl groups or oxygen at the C3′ position can be the most active compounds. This observation suggests that these types of acyl groups might be important to the enhanced anti-HIV activity.

The invention is also directed to a method for treating a subject infected with HIV-1 by administering at least one of the above-noted triterpene derivatives, optionally in combination with any one or more of the known anti-AIDS therapeutics or an immunostimulant.

Other features, advantages, embodiments, aspects and objects of the present invention will be clear to those skilled in the areas of relevant art, based upon the description, teaching and guidance presented herein.

The analogs of the present invention can have anti-retroviral activity, thus providing suitable compounds and compositions for treating retroviral infections, optionally with additional pharmaceutically active ingredients, such as anti-retroviral, anti-HIV, and/or immuno-stimulating compounds or antiviral antibodies or fragments thereof.

By the term “anti-retroviral activity” or “anti-HIV activity” is intended the ability to inhibit at least one of:
(1) viral pro-DNA integration into host cell genome;
(2) retroviral attachment to cells;
(3) viral entry into cells;
(4) cellular metabolism which permits viral replication;
(5) inhibition of intercellular spread of the virus;
(6) synthesis and/or cellular expression of viral antigens;
(7) viral budding or maturation;
(8) activity of virus-coded enzymes (such as reverse transcriptase, integrase and proteases); and/or
(9) any known retroviral or HIV pathogenic actions, such as, for example, immunosuppression. Thus, any activity which tends to inhibit any of these mechanisms is “anti-retroviral activity” or “anti-HIV activity.”

A triterpene derivative of the present invention can be used for treatment of retroviral (e.g., HIV) infection either alone, or in combination with other modes of therapy known in the art. Such modes of therapy can include chemotherapy with drugs, such as, but not limited to, at least one of AZT, 3TC, ddC, d4T, ddI, tenofovir, abacavir, nevirapine, delavirdine, emtricitabine, efavirenz, saquinavir, ritonavir, indinavir, nelfinavir, lopinavir, amprenavir, fosamprenavir, and atazanavir or any other antiretroviral drugs or antibodies in combination with each other, or associated with a biologically based therapeutic, such as, for example, gp41-derived peptides enfuvirtide (Fuzeon; Timeris-Roche) and T-1249 (Trimeris), or soluble CD4, antibodies to CD4, and conjugates of CD4 or anti-CD4, or as additionally presented herein.

A triterpene derivative according to the present invention can be used in treating blood products, such as those maintained in blood banks. The nation's blood supply is currently tested for antibodies to HIV. However, the test is still imperfect and samples which yield negative tests can still contain HIV virus. Treating the blood and blood products with the triterpene derivatives of the present invention can add an extra margin of safety by reducing or eliminating activity of any retrovirus that may have gone undetected.

In addition, triterpene derivatives of the present invention can be used as prophylactics to prevent transmission of HIV infection between individuals. For example, the derivatives can be administered orally or by injection to an HIV infected pregnant woman and/or fetus during pregnancy or immediately prior to, at, or subsequent to birth, to reduce the probability that the newborn infant becomes infected. Also, the derivatives can be administered vaginally immediately prior to childbirth to prevent infection of the infant during passage through the birth canal. Further, the derivatives of the present invention can be used during sexual intercourse to prevent transmission of HIV by applying a retroviral inhibiting effective amount of a topical composition including one or more compounds of Formulae I, II, III, IV, V or VI to vaginal or other mucosa prior to sexual intercourse. For example, the derivatives of the present invention can be used to prevent transmission of HIV from an infected male to an uninfected female or vice versa.

Pharmaceutical Compositions

Pharmaceutical compositions of the present invention can comprise at least one triterpene derivative. Pharmaceutical compositions according to the present invention can also further comprise other anti-viral agents such as, but not limited to, AZT (zidovudine, RETROVIR, GlaxoSmithKline), 3TC (lamivudine, EPIVIR®, GlaxoSmithKline), AZT+3TC, (COMBIVIR®, GlaxoSmithKline) AZT+3TC+abacvir (TRIZIVIR®, GlaxoSmithKline), ddI (didanosine, VIDEX®, Bristol-Myers Squibb), ddC (zalcitabine, HIVID®, Hoffmann-LaRoche), D4T (stavudine, ZERIT®, Bristol-Myers Squibb), abacavir (ZIAGEN®, GlaxoSmithKline), nevirapine (VIRAMUNE®, Boehringher Ingelheim), delavirdine (Pfizer), efavirenz (SUSTIVA®, DuPont Pharmaceuticals), tenofovir (VIREAD®, Gilead Sciences), saquinavir (INVIRASE®, FORTOVASE®, Hoffmann-La Roche), ritonavir (NORVIR®, Abbott Laboratories), indinavir (CRIXIVAN®, Merck and Company), nelfinavir (VIRACEPT®, Pfizer), amprenavir (AGENERASE®, GlaxoSmithKline), adefovir (PREVEON®, HEPSERA®, Gilead Sciences), atazanavir (REYATAZ®, Bristol-Myers Squibb), fosamprenavir (LEXIVA®, GlaxoSmithKline) and hydroxyurea (HYDREA®, Bristol-Meyers Squibb), or any other antiretroviral drugs or antibodies in combination with each other, or associated with a biologically based therapeutic, such as, for example, gp41-derived peptides enfuvirtide (FUZEON®, Roche and Trimeris) and T-1249, or soluble CD4, antibodies to CD4, and conjugates of CD4 or anti-CD4, or as additionally presented herein.

Additional suitable anti-viral agents for optimal use with a triterpene derivative of the present invention can include, but is not limited to, amphotericin B (FUNGIZONE®); Ampligen (mismatched RNA; Hemispherx Biopharma); BETASERON® (β-interferon, Chiron); butylated hydroxytoluene; Carrosyn (polymannoacetate); Castanospermine; Contracan (stearic acid derivative); Creme Pharmatex (containing benzalkonium chloride); 5-unsubstituted derivative of zidovudine; penciclovir (DENAVIR®, Novartis); famciclovir (FAMVIR®, Novartis); acyclovir (ZOVIRAX®, GlaxoSmithKline); cytofovir (VISTIDE®, Gilead); ganciclovir (CYTOVENE®, Hoffman LaRoche); dextran sulfate; D-penicillamine (3-mercapto-D-valine); FOSCARNET® (trisodium phosphonoformate; AstraZeneca); fusidic acid; glycyrrhizin (a constituent of licorice root); HPA-23 (ammonium-21-tungsto-9-antimonate); ORNIDYL® (eflornithine, Aventis); nonoxynol; pentamidine isethionate (PENTAM-300); Peptide T (octapeptide sequence, Peninsula Laboratories); Phenytoin (Pfizer); INH or isoniazid; ribavirin (VIRAZOLE®, Valeant Pharmaceuticals); rifabutin, ansamycin (MYCOBUTIN®, Pfizer); CD4-IgG2 (Progenics Pharmaceuticals) or other CD4-containing or CD4-based molecules; Trimetrexate (Medimmune); suramin and analogues thereof (Bayer); and WELLFERON® (α-interferon, GlaxoSmithKline).

Pharmaceutical compositions of the present invention can also further comprise immunomodulators. Suitable immunomodulators for optional use with a triterpene derivative of the present invention in accordance with the present invention can include, but are not limited to: ABPP (Bropririmine); anti-human interferon-α-antibody; ascorbic acid and derivatives thereof; interferon-β; Ciamexon; cyclosporin; cimetidine; CL-246,738; colony stimulating factors, including GM-CSF; dinitrochlorobenzene; HE2000 (Hollis-Eden Pharmaceuticals); inteferon-γ; glucan; hyperimmune gamma-globulin (Bayer); immuthiol (sodium diethylthiocarbamate); interleukin-1 (Hoffmann-LaRoche, Amgen), interleukin-2 (IL-2) (Chiron); isoprinosine (inosine pranobex); Krestin; LC-9018 (Yakult); lentinan (Yamanouchi); LF-1695; methionine-enkephalin; Minophagen C; muramyl tripeptide, MTP-PE; naltrexone (Barr Laboratories); RNA immunomodulator; REMUNE® (Immune Response Corporation); RETICULOSE® (Advanced Viral Research Corporation); shosaikoto; ginseng; thymic humoral factor; Thymopentin; thymosin factor 5; thymosin 1 (ZADAXIN®, SciClone); thymostimulin; TNF (tumor necrosis factor, Genentech); and vitamin preparations.

In some embodiments, the animal subject of the present invention is a mammal. By the term “mammal” is meant an individual belonging to the class Mammalia. The invention is particularly useful in the treatment of human patients.

The term “treating” means the administering to subjects a triterpene derivative for purposes which can include prevention, amelioration, or cure of a retroviral-related pathology.

Medicaments are considered to be provided “in combination” with one another if they are provided to the patient concurrently or if the time between the administration of each medicament is such as to permit an overlap of biological activity.

In some embodiments, at least one triterpene derivative comprises a single pharmaceutical composition.

Pharmaceutical compositions for administration according to the present invention can comprise at least one triterpene derivative according to the present invention in a pharmaceutically acceptable form optionally combined with a pharmaceutically acceptable carrier. These compositions can be administered by any means that achieve their intended purposes. Amounts and regimens for the administration of a triterpene derivative according to the present invention can be determined readily by those with ordinary skill in the clinical art of treating a retroviral pathology.

For example, administration can be by parenteral, such as subcutaneous, intravenous, intramuscular, intraperitoneal, transdermal, or buccal routes. Alternatively, or concurrently, administration can be by the oral route. The dosage administered depends upon the age, health and weight of the recipient, type of previous or concurrent treatment, if any, frequency of treatment, and the nature of the effect desired.

Compositions within the scope of this invention include all compositions comprising at least one triterpene derivative according to the present invention in an amount effective to achieve its intended purpose. While individual needs vary, determination of optimal ranges of effective amounts of each component is within the skill of the art. Typical dosages comprise about 0.1 mg/kg to about 100 mg/kg body weight. In some embodiments, the dosages comprise about 1 mg/kg to about 100 mg/kg body weight of the active ingredient. In some embodiments, the dosages comprise about 2.5 mg/kg to about 50 mg/kg body weight. In some embodiments, the dosages comprise about 5 mg/kg to about 25 mg/kg body weight.

Therapeutic administration can also include prior, concurrent, subsequent or adjunctive administration of at least one additional triterpene derivative according to the present invention or other therapeutic agent, such as an anti-viral or immune stimulating agent. In such an approach, the dosage of the second drug can be the same as or different from the dosage of the first therapeutic agent. In some embodiments, the drugs are administered on alternate days in the recommended amounts of each drug.

Administration of a compound of the present invention can also optionally include previous, concurrent, subsequent or adjunctive therapy using immune system boosters or immunomodulators. In addition to the pharmacologically active compounds, a pharmaceutical composition of the present invention can also contain suitable pharmaceutically acceptable carriers comprising excipients and auxiliaries which facilitate processing of the active compounds into preparations which can be used pharmaceutically. In some embodiments, the preparations, particularly those preparations which can be administered orally and which can be used in the above-described type of administration, such as tablets, dragees, and capsules, and also preparations which can be administered rectally, such as suppositories, as well as suitable solutions for administration by injection or orally, contain from about 1 percent to about 99 percent, preferably from about 20 percent to about 75 percent of active compound(s), together with the excipient.

Pharmaceutical preparations of the present invention are manufactured in a manner which is itself known, for example, by means of conventional mixing, granulating, dragee-making, dissolving, or lyophilizing processes. Thus, pharmaceutical preparations for oral use can be obtained by combining the active compounds with solid excipients, optionally grinding the resulting mixture, and processing the mixture of granules, after adding suitable auxiliaries, if desired or necessary, to obtain tablets or dragee cores.

Suitable excipients are, e.g., fillers such as saccharide, for example, lactose or sucrose, mannitol or sorbitol; cellulose preparations and/or calcium phosphates, such as tricalcium phosphate or calcium hydrogen phosphate; as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents can be added such as the above-mentioned starches and also carboxymethyl starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions can be used, which can optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl cellulose phthalate are used. Dyestuffs or pigments can be added to the tablets or dragee coatings, for example, for identification or in order to characterize combinations of active compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which can be mixed with fillers such as lactose, binders such as starches, and/or lubricants such as talc or magnesium stearate and, optionally, stabilizers. In some embodiments using soft capsules, the active compounds are dissolved or suspended in suitable liquids, such as fatty oils or liquid paraffin. In addition, stabilizers can be added.

Possible pharmaceutical preparations which can be used rectally include, for example, suppositories which consist of a combination of the active compounds with a suppository base. Suitable suppository bases are, for example, natural or synthetic triglycerides, or paraffin hydrocarbons. In addition, it is also possible to use gelatin rectal capsules which consist of a combination of the active compounds with a base. Possible base materials include, for example, liquid triglycerides, polyethylene glycols, or paraffin hydrocarbons.

Suitable formulations for parenteral administration include aqueous solutions of the active compounds in water-soluble form, for example, water-soluble salts. In addition, suspensions of the active compounds as appropriate oily injection suspensions can be administered. Suitable liphophilic solvents or vehicles include fatty oils, such as sesame oil, or synthetic fatty acid esters, such as ethyl oleate or triglycerides. Aqueous injection suspensions that can contain substances which increase the viscosity of the suspension include, for example, sodium carboxymethyl cellulose, sorbitol, and/or dextran. Optionally, the suspension can also contain stabilizers.

A pharmaceutical formulation for systemic administration according to the invention can be formulated for enteral, parenteral or topical administration. Indeed, all three types of formulation can be used simultaneously to achieve systemic administration of the active ingredient.

Suitable formulations for oral administration include hard or soft gelatin capsules, dragees, pills, tablets, including coated tablets, elixirs, suspensions, syrups or inhalations and controlled release forms thereof.

Solid dosage forms in addition to those formulated for oral administration include rectal suppositories.

Prophylactic topical compositions for preventing HIV infection between individuals during childbirth or sexual intercourse include one or more compounds of Formulae I, II, III, IV, V and VI and at least one pharmaceutically acceptable topical carrier or diluent. The topical composition can be, for example, in the form of an ointment, a cream, a gel, a lotion, a paste, a jelly, a spray, a foam, or a sponge. The dosage amount of a compound of Formulae I, II, III, IV, V and VI in a prophylactic topical formulation is, in general, less than about 1,000 milligrams, and in some embodiments from about 0.01 milligrams to about 100 milligrams. The topical formulations can include other prophylactic ingredients. The carrier and diluents should be acceptable in the sense of being compatible with other ingredients of the formulation and not deleterious to the recipient.

Topical prophylactic formulations include those suitable for vaginal, rectal or topical administration. The formulations can, where appropriate, be conveniently presented in discrete dosage units, and can be prepared by any of the methods known in the art of pharmacy. All such methods include the step of bringing the active agent into association with liquid carriers, gels or finely divided solid carriers or both and then, if necessary, shaping the product into the desired formulation.

Prophylactic formulations suitable for vaginal administration can be presented as pessaries, tampons, creams, gels, pastes, jelly, foams, or sprays, or aqueous or oily suspensions, solutions or emulsions (liquid formulations) containing suitable carriers known in the art in addition to the active agent. Liquid formulations can contain conventional additives, such as, suspending agents, emulsifying agents, non-aqueous vehicles including edible oils, or preservatives. These formulations are useful to prevent both sexual transmission of HIV and infection of an infant during passage through the birth canal. In one example, the vaginal administration can take place prior to sexual intercourse, or immediately prior to childbirth.

In some embodiments, prophylactic formulations suitable for rectal or vaginal administration having a solid carrier are represented as unit dose suppositories. Suitable carriers include cocoa butter and other materials commonly used in the art. Suppositories can be formed, for example, mixing one or more compounds of Formulae I, II, III, IV, V and VI with one or more softened or melted carriers followed by chilling and shaping in molds.

Prophylactic formulations according to the invention can also be in the form of drops formulated with an aqueous or non-aqueous base comprising one or more dispersing agents, solubilizing agents, or suspending agents. Liquid sprays can be delivered from pressurized packs.

Prophylactic formulations according to the invention can be adapted to give sustained delivery. Also, the prophylactic formulations can include other active agents, such as spermicidal agents, antimicrobial agents, and anti-viral agents.

The triterpene derivatives of the present invention can also be administered in the form of an implant when compounded with a biodegradable slow-release carrier. Alternatively, the triterpene derivatives of the present invention can be formulated as a transdermal patch for continuous release of the active ingredient.

Suitable formulations for topical administration include creams, gels, jellies, mucilages, pastes and ointments. Suitable injectable solutions include intravenous subcutaneous and intramuscular injectable solutions. Alternatively, the triterpene derivatives can be administered in the form of an infusion solution or as a nasal inhalation or spray.

The compounds of the present invention can be prepared using methods known to those skilled in the art. Various triterpene backbones can be obtained from commercial sources. The compounds of FIG. I of the present invention can be prepared in a manner similar to that exemplified by the modification of betulin as shown in Scheme 1. Betulin or dihydrobetulin can be heated overnight at 95° C. with 6-fold of the appropriate anhydride in anhydrous pyridine in the presence of 4-(dimethylamino)pyridine (DMAP). CH₂OR_{z} corresponds to R₁₁ as defined above for A(ii). When thin layer chromatography (TLC) indicates complete consumption of starting material, the reaction can be diluted with EtOAc and washed with 10% HCl solution. The EtOAc layer can then be dried over MgSO₄ and subjected to column chromatography.

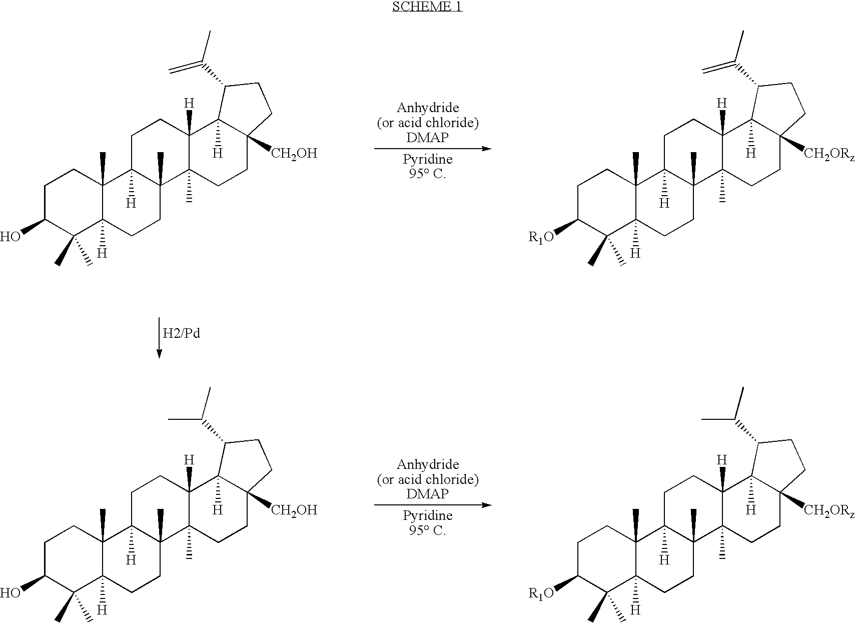

The compounds of FIG. I of the present invention can be prepared in a manner similar to that exemplified by the modification of betulin as shown in Scheme 2. Scheme 2 depicts the synthesis route for compounds where R₁ and R₁₁ are substituted or unsubstituted carboxyacyl. CH₂OR_{z} corresponds to R₁₁ defined above for A(ii).

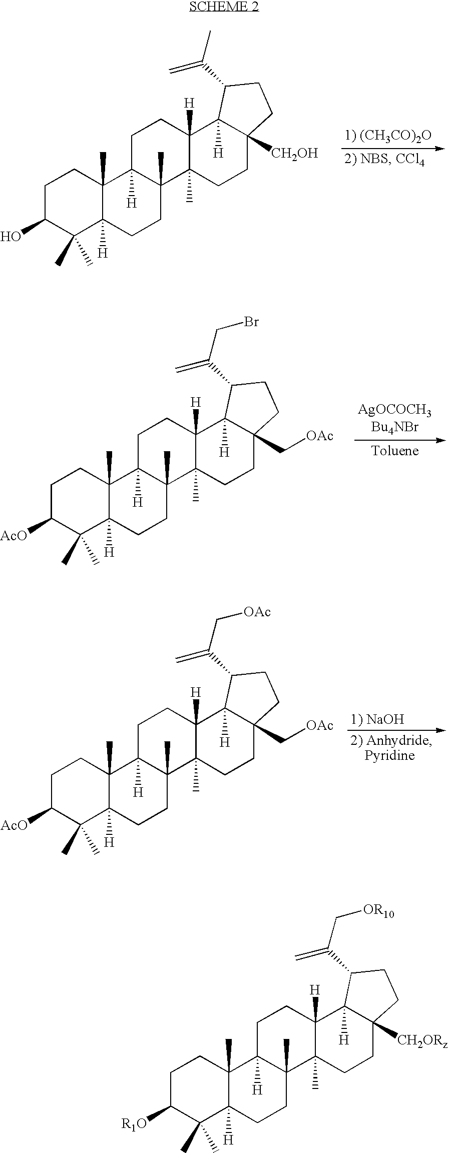

Scheme 3 depicts an alternative method of synthesizing the compounds of the present invention by the use of solid phase organic synthesis (Pathak, A., et al. Combinatorial Chem. and High Throughput Screening 5, 241-248 (2002)). Briefly, a triterpene backbone can be linked to a resin via ester or amide bond formation at R₅, R₁₁ or R₁₃ (denoted by Rₐ). Any resin which allows cleavage of compounds under mild conditions can be used, e.g., 2-chlorotrityl chloride resin or Sieber amide resin. An amino acid can be introduced as a spacer between the triterpene and the resin if desired. Once the triterpene is immobilized onto the resin scaffold, diversity can be introduced as desired at the C3 position by adding the acid form of the desired R₁ substituents (denoted by R_{b}).

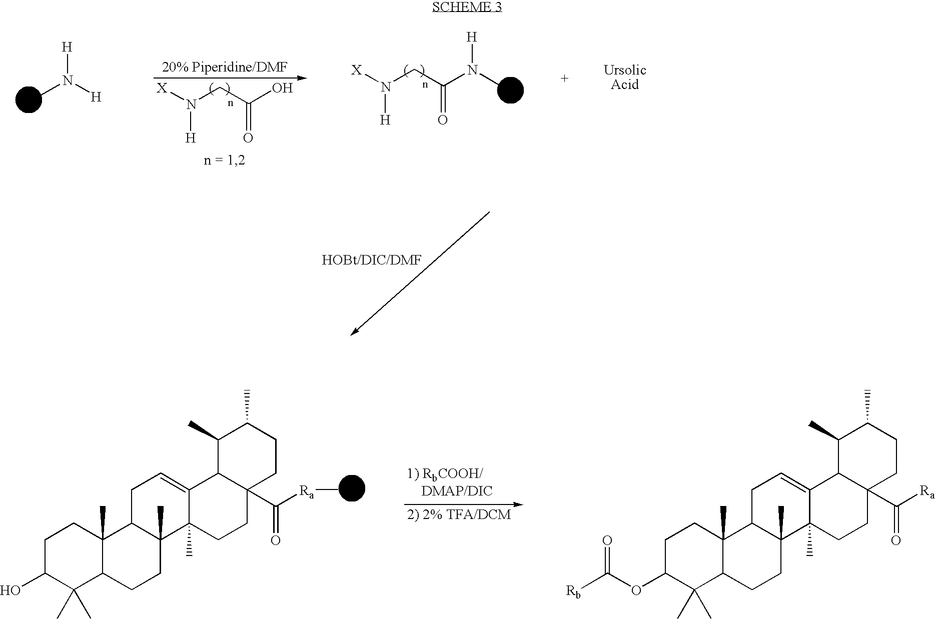

The triterpene derivatives of the present invention can be prepared as shown in Scheme 4. Protection of the 28-hydroxyl group of betulin (1) with triphenylmethyl ether group yields betulin 28-O-triphenylmethyl ether (2), whose solution in pyridine is further treated with an appropriate dicarboxylic acid in the presence of dimethylamino pyridine at reflux. Finally, the 28-protective group is removed by refluxing with pyridium p-toluenesulfonate in CH₂Cl₂-EtOH to give desired 3-O-acyl betulin derivatives.

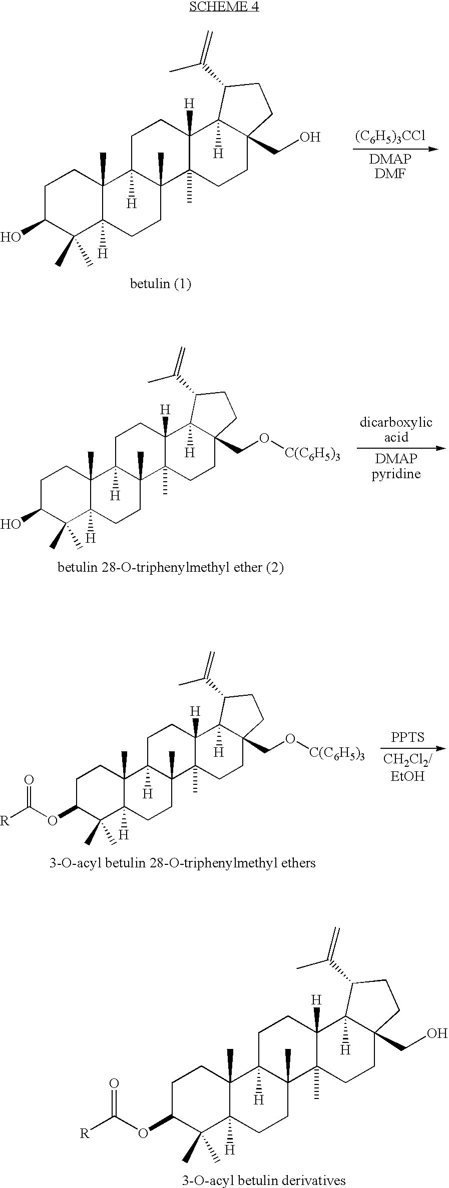

The biological evaluation of HIV-1 inhibition can be carried out as follows according to established protocols (Montefiori, D. C., et al., Clin. Microbiol 26, 231-235 (1988)). The human T-cell line, MT-2, is maintained in continuous culture with complete medium (RPMI 1640 with 10% fetal calf serum supplemented with L-glutamine at 5% CO₂ and 37° C.). Test samples are first dissolved in dimethyl sulfoxide at a concentration of 10 mg/ml to generate master stocks with dilutions made into tissue culture media to generate working stocks. The following drug concentrations are routinely used for screening: 100, 20, 4 and 0.8<g/ml. For agents found to be active, additional dilutions are prepared for subsequent testing so that an accurate EC₅₀ value (defined below) can be determined. Test samples are prepared and to each sample well is added 90<1 of media containing MT-2 cells at 3×10⁵cells/ml and 45<1 of virus inoculum (HIV-1 IIIIB isolate) at a concentration necessary to result in 80% killing of the cell targets at 5 days post-infection (PI). Control wells containing virus and cells only (no drug) and cells only (no virus or drug) are also prepared. A second set of samples are prepared identical to the first and are added to cells under identical conditions without virus (mock infection) for toxicity determinations (IC₅₀ defined below). In addition, AZT is also assayed during each experiment as a positive drug control. On day 5 PI, virus-induced cell killing is determined by measuring cell viability using the XTT method. Compound toxicity is determined by XTT using the mock-infected samples. If a test sample has suppressive capability and is not toxic, its effects are reported in the following terms: IC₅₀, the concentration of test sample which is toxic to 50% of the mock-infected MT-2 cells; EC₅₀, the concentration of the test sample that is able to suppress HIV replication by 50%; and the Therapeutic index (TI) the ratio of the IC₅₀ to EC₅₀.

The following example is illustrative, but not limiting, of the method and compositions of the present invention. Other suitable modifications and adaptations of the variety of conditions and parameters normally encountered and obvious to those skilled in the art are within the spirit and scope of the invention.

Those skilled in the art will recognize that while specific embodiments have been illustrated and described, various modifications and changes can be made without departing from the spirit and scope of the invention.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims. All publications, patent applications and patents cited herein are fully incorporated by reference.

1. A compound of Formula I: 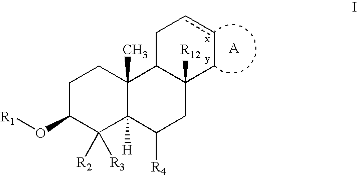
or a pharmaceutically acceptable salt or ester thereof;
wherein A is a fused ring of formula 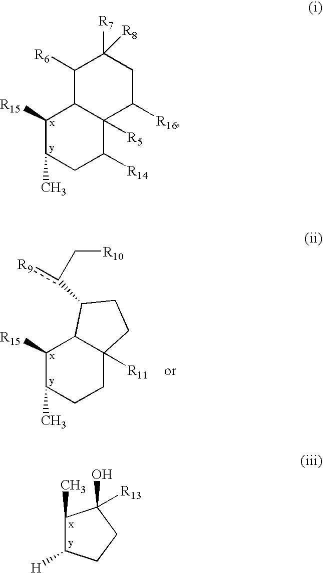
wherein the ring carbons designated x and y in the formulas of A are the same as the ring carbons designated x and y in Formula I;
R₁ is selected from the group consisting of 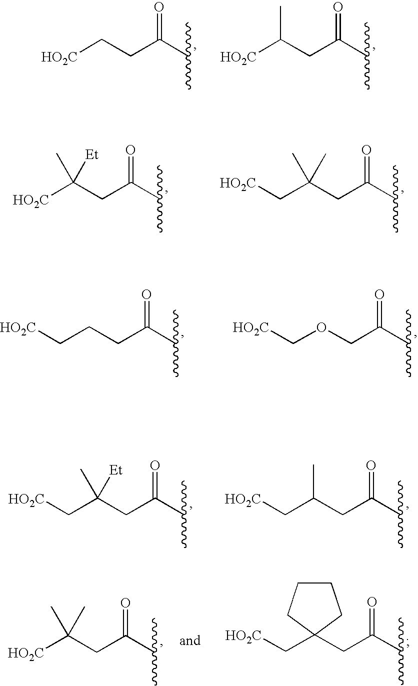
R₂ and R₃ are independently hydrogen, methyl, halogen, hydroxyl, carboxyl or —COOR₁₇;
R₄ is hydrogen, methyl, halogen, or hydroxyl;
R₅ is carboxyalkoxycarbonyl, alkoxycarbonyl, alkanoyloxymethyl, carboxyalkanoyloxymethyl, alkoxymethyl, carboxyalkoxymethyl, aminocarbonyl, alkylaminocarbonyl, dialkylaminocarbonyl, alkoxyalkylaminocarbonyl, alkoxyalkoxyalkylaminocarbonyl, alkoxycarbonylaminoalkoxyalkylaminocarbonyl, alkoxycarbonylaminoalkylaminocarbonyl, alkylcarbonylaminoalkylaminocarbonyl, aminoalkylaminocarbonyl, aminoalkoxyalkylaminocarbonyl, monoalkylaminoalkylaminocarbonyl, dialkylaminoalkylaminocarbonyl, heterocyclylcarbonyl, heterocyclylalkylaminocarbonyl, cycloalkylaminocarbonyl, arylaminocarbonyl, arylalkylaminocarbonyl, arylcarbonylaminoalkylaminocarbonyl, or heteroarylaminocarbonyl, any of which is optionally substituted by one or more hydroxyl or halo, or R₅ is a carboxyl or hydroxymethyl, or when either R₂ or R₃ are carboxyl, then R₅ can be methyl;
R₆ is hydrogen, methyl, hydroxyl or halogen;
R₇ and R₈ are independently hydrogen or C₁₋₆ alkyl;
R₉ is CH₂ or CH₃;
R₁₀ is hydrogen, hydroxyl or methyl;
R₁₁ is methyl, methoxycarbonyl, carboxyalkoxycarbonyl, alkanoyloxymethyl, alkoxymethyl or carboxyalkoxymethyl, any of which is optionally substituted by one or more hydroxyl or halo;
R₁₂ is hydrogen or methyl;
R₁₃ is hydrogen or methyl;
R₁₄ is hydrogen or hydroxyl;
R₁₅ is hydrogen if C12 and C13 form a single bond, or R₁₅ is absent if C12 and C13 form a double bond;
R₁₆ is hydrogen or hydroxyl;
R₁₇ is alkyl or carboxyalkyl, where the alkyl chain can be optionally substituted by one or more hydroxyl or halo, or can be interrupted by a nitrogen, sulfur or oxygen atom, or combinations thereof; and
wherein the straight dashed line represents an optional double bond between C12 and C13 or C20 and C29;
with the proviso that when A is 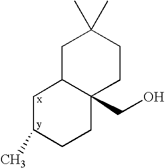
then R₁ cannot be glutaryl or succinyl when a double bond exists between C12 and C13;
when A is (ii) and R₁₁ is methyl, then R₁ cannot be succinyl;
when A is (iii) and R₂, R₃ and R₁₃ are each hydrogen, then R₁ cannot be succinyl; and
with the proviso that A (i) cannot be 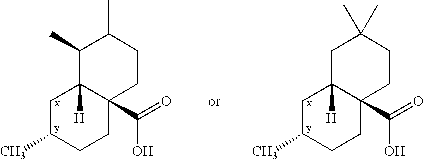
when R₂ and R₃ are both methyl and a double bond exists between C12 and C13.

2. The compound of claim 1, wherein R₂ and R₃ are both methyl.

3. The compound of claim 1, wherein R₁ is 3′,3′-dimethylsuccinyl.

4. The compound of claim 1, wherein A is (i) and R₅ is in the β position.

5. The compound of claim 1, wherein A is (i) and R₆ is in the β position.

6. The compound of claim 1, wherein A is (i) and R₁₄ is in the α position.

7. The compound of claim 1, wherein A is (i), R₇ is α-methyl, and R₈ is hydrogen.

8. The compound of claim 1, wherein A is (i), R₈ is α-methyl, and R₇ is hydrogen.

9. The compound of claim 1, wherein A is (i) and both R₇ and R₈ are methyl.

10. The compound of claim 1, wherein A is (ii) and R₁₁ is in the βposition.

11. A compound of claim 1, having Formula II: 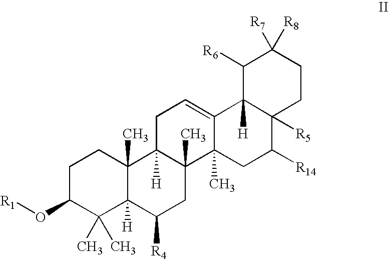
wherein R₁, R₄, R₅, R₆, R₇, R₈ and R₁₄ are as defined in claim 1.

12. A compound of claim 11, wherein R₆ is β-methyl, R₈ is hydrogen, R₅ is hydroxymethyl and R₁ is 3′,3′-dimethylglutaryl, 3′,3′-dimethylsuccinyl, glutaryl or succinyl.

13. A compound of claim 11, wherein R₅ is hydroxymethyl, —CO₂(CH₂)ₙCOOH or —CO₂(CH₂)ₙCH₃ and n is 0-6.

14. A compound of claim 11, wherein R₅ is —COC(O)(CH₂)ₙCH₃ or —COC(O)(CH₂)ₙCOOH and n is 0-6.

15. A compound of claim 11, wherein R₅ is —CO(CH₂)ₙCH₃ or —CO(CH₂)ₙCOOH and n is 0-6.

16. The compound of claim 11, which is one of:
3-O-(3′,3′-dimethylsuccinyl)uvaol;
3-O-(3′,3′-dimethylsuccinyl)erythrodiol;
3-O-(3′,3′-dimethylsuccinyl)echinocystic acid; and
3-O-(3′,3′-dimethylsuccinyl)sumaresinolic acid.

17. A compound of claim 1, having Formula III: 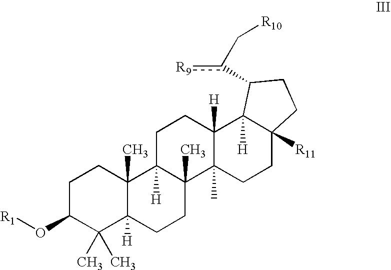
wherein R₁, R₉, R₁₀, and R₁₁ are as defined in claim 1.

18. A compound of claim 17, wherein R₁ is 3′,3′-dimethylglutaryl, 3′,3′-dimethylsuccinyl, glutaryl or succinyl.

19. A compound of claim 17, wherein R₁₁ is methyl, carboxyalkoxycarbonyl, alkanoyloxymethyl, alkoxymethyl, or carboxyalkoxymethyl.

20. A compound of claim 17, wherein R₁ is methyl or —CO₂(CH₂)ₙCOOH and n is 0-6.

21. A compound of claim 17, wherein R₁₁ is —COC(O)(CH₂)ₙCH₃ and n is 0-6.

22. A compound of claim 17, wherein R₁₁ is —CO(CH₂)ₙCH₃ or —CO(CH₂)ₙCOOH and n is 0-6.

23. The compound of claim 17, which is one of:
3-O-(3′,3′-dimethylsuccinyl)lupeol;
3-O-(3′,3′-dimethylsuccinyl)dihydrolupeol;
3-O-(3′,3′-dimethylsuccinyl)-17β-methylester-betulinic acid; and
3-O-(3′,3′-dimethylsuccinyl)-17β-methylester-dihydrobetulinic acid.

24. A compound of claim 1, having Formula IV: 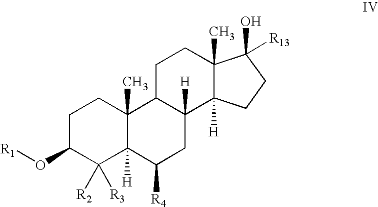
wherein R₁, R₂, R₃, R₄, and R₁₃ are as defined in claim 1.

25. A compound of claim 24, wherein R₁ is 3′,3′-dimethylglutaryl, 3′,3′-dimethylsuccinyl, glutaryl or succinyl.

26. A compound of claim 24, wherein R₁ is 3′,3′-dimethylglutaryl, 3′,3′-dimethylsuccinyl, glutaryl or succinyl, and both R₂ and R₃ are methyl.

27. The compound of claim 24, which is one of:
3-O-(3′,3′-dimethylsuccinyl)-4,4-dimethylandrostanediol;
3-O-(3′,3′-dimethylsuccinyl)-17α-methylandrostanediol; and
3-O-(3′,3′-dimethylsuccinyl)androstanediol.

28. A compound of claim 1, having Formula V: 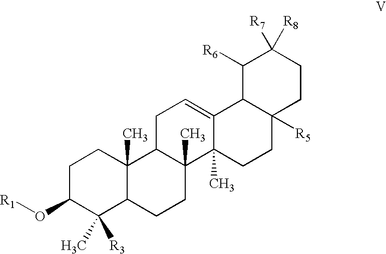
wherein R₁ R₃, R₅, R₆, R₇, and R₈ are as defined in claim 1.

29. A compound of claim 28, wherein R₆ is hydrogen, R₇ is methyl, and R₈ is methyl.

30. A compound of claim 28, wherein R₆ is methyl, R₇ is hydrogen and R₈ is methyl.

31. The compound of claim 28, which is one of:
3-O-(3′,3′-dimethylsuccinyl)-α-boswellic acid; and
3-O-(3′,3′-dimethylsuccinyl)-β-boswellic acid.

32. A compound of claim 1, having Formula VI: 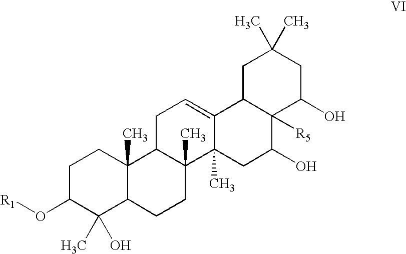
wherein R₁ and R₅ are as defined in claim 1.

33. A compound of claim 32, which is 3-O-(3′,3′-dimethylsuccinyl)gymnemic acid.

34. A pharmaceutical composition, comprising a compound according to claim 1 or a pharmaceutically acceptable ester or salt thereof, and a pharmaceutically acceptable carrier.

35. A pharmaceutical composition according to claim 34, further comprising an anti-viral agent or an immunostimulating agent.

36. A pharmaceutical composition according to claim 35, wherein said antiviral agent is selected from the group consisting of one or more of zidovudine, lamivudine, zalcitabine, stavudine, didanosine, tenofovir, abacavir, nevirapine, delavirdine, emtricitabine, efavirenz, saquinavir, ritonavir, indinavir, nelfinavir, lopinavir, amprenavir, fosamprenavir, atazanavir, enfuvirtide, hydroxyurea, interleukin-2, gamma globulin, amantadine, guanidine hydroxybenzimidazole, interferon-α, interferon-β, interferon-γ, a thiosemicarbazone, methisazone, rifampin, ribavirin, a pyrimidine analog, a purine analog, foscarnet, phosphonoacetic acid, acyclovir, a dideoxynucleoside, and ganciclovir.

37. A method for inhibiting a retroviral infection in cells or tissue of an animal comprising administering an effective retroviral inhibiting amount of a pharmaceutical composition according to claim 34.

38. The method according to claim 37, wherein said composition is administered to provide said compound in an amount ranging from about 0.1 to about 100 mg/kg body weight.

39. The method according to claim 38, wherein said composition is administered to provide said compound in an amount ranging from about 5 to about 25 mg/kg body weight.

40. The method according to claim 39, wherein said animal is a human.

41. A pharmaceutical composition comprising a compound according to claims 11, 17, 24, 28 or 32 or a pharmaceutically acceptable ester or salt thereof, and a pharmaceutically acceptable carrier.

42. A pharmaceutical composition according to claim 41 further comprising a drug selected from an anti-viral agent or an immunostimulating agent.

43. A pharmaceutical composition according to claim 42, wherein said antiviral agent is selected from the group consisting of one or more of zidovudine, lamivudine, zalcitabine, stavudine, didanosine, tenofovir, abacavir, nevirapine, delavirdine, emtricitabine, efavirenz, saquinavir, ritonavir, indinavir, nelfinavir, lopinavir, amprenavir, fosamprenavir, atazanavir, enfuvirtide, hydroxyurea, interleukin-2, gamma globulin, amantadine, guanidine hydroxybenzimidazole, interferon-α, interferon-β, interferon-γ, a thiosemicarbazone, methisazone, rifampin, ribavirin, a pyrimidine analog, a purine analog, foscarnet, phosphonoacetic acid, acyclovir, a dideoxynucleoside, and ganciclovir.

44. A method for inhibiting a retroviral infection in cells or tissue of an animal comprising administering an effective retroviral inhibiting amount of a pharmaceutical composition according to claim 43.

45. The method according to claim 44, wherein said composition is administered to provide said compound in an amount ranging from about 0.1 to about 100 mg/kg body weight.

46. The method according to claim 45, wherein said composition is administered to provide said compound in an amount ranging from about 5 to about 25 mg/kg body weight.

47. The method according to claim 46, wherein said animal is a human.

48. A method of inhibiting a retroviral infection by contacting a cell with a compound of claims 1, 11, 17, 24, 28 or 32.

49. A method of preventing transmission of HIV infection from an HIV infected pregnant woman to a fetus, comprising administering to said woman and/or said fetus a retroviral inhibiting effective amount of a compound of claim 1, 11, 17, 24, 28 or 32 during pregnancy or immediately prior to, at, or subsequent to birth.

50. A method of preventing transmission of HIV infection during sexual intercourse, comprising applying a retroviral inhibiting effective amount of one or more compounds of claim 1, 11, 17, 24, 28 or 32 to vaginal or other mucosa prior to sexual intercourse.
